(19) United States
(12) Patent Application Publication
(10) Pub. No.: US 2009/0319020 A1
(43) Pub. Date: Dec. 24, 2009

(54) DEVICES, SYSTEMS, AND METHODS FOR PROMOTING ENDOTHELIALIZATION

(75) Inventor: Ghassan S. Kassab, Zionsville, IN (US)

Correspondence Address: ICE MILLER LLP, ONE AMERICAN SQUARE, SUITE 3100, INDIANAPOLIS, IN, 46282-0200, US

(73) Assignee: DTHERAPEUTICS, LLC, Zionsville, IN (US)

(21) Appl. No.: 12/521,196

(22) PCT Filed: Jan. 22, 2008
(86) PCT No.: PCT/US08/00763

§ 371 Date: Jun. 25, 2009

Related U.S. Application Data
(60) Provisional application No. 60/881,834, filed on Jan. 23, 2007.

(51) Int. Cl. A61F 2/06

(52) U.S. Cl. 623/1.11; 623/1.15; 623/1.46

(57) ABSTRACT

Devices, systems, and methods for promoting endothelialization and preventing restenosis are disclosed. At least some of the embodiments disclosed herein promote endothelialization and prevent restenosis by reducing local blood flow turbulence. At least some of the embodiments disclosed herein promote endothelialization and prevent restenosis by directing endothelial progenitor cells to a targeted site using magnetic and other means.

This International Patent Application claims priority to U.S. Provisional Patent Application Serial No. 60/881,834, filed Jan. 23, 2007, the contents of which are incorporated herein by reference.

### BACKGROUND

### Coronary Heart Disease

Coronary heart disease, also known as ischemic heart disease, is a leading cause of death in the United States and throughout the world. It is generally characterized by a diminished blood flow to the myocardium caused by the narrowing of the coronary arteries due to development of atherosclerotic plaque. If left untreated, the disease can lead to chronic heart failure, which can be defined as a significant decrease in the heart's ability to pump blood, and myocardial infarction.

Coronary heart disease can be treated using intra-coronary stents, often in combination with percutaneous transluminal coronary angioplasty. Such stents are used to prevent arterial dissection caused by angioplasty alone and to help maintain an increased coronary lumen diameter (and therefore improve blood flow to the cardiac tissue) by, inter alia, preventing vessel recoil. In addition, stents can minimize restenosis by diminishing the incidence of intimal hyperplasia. Intimal hyperplasia refers to the growth of smooth muscle tissue within the vessel lumen. It is caused, at least in part, by disturbed blood flow and turbulence at the site of the stent, as well as low wall shear stresses, and it is a problem particularly with smaller diameter vessels. Smaller diameter vessels (e.g., less than about 4 millimeters in diameter) are generally more difficult to successfully stent than larger diameter vessels (e.g., coronary arteries).

While stents are most commonly used to prevent restenosis in coronary arteries, they may also be used in other body lumens, including other arteries and veins, an esophagus, a colon, a bile duct, a trachea or other respiratory duct, and various lumens of the urinary tract. In addition, certain stents may be placed in an artery to strengthen a weakened portion of the arterial wall (i.e., to treat an aneurysm).

### Endothelialization

The interaction of cells with extracellular matrix molecules plays a crucial role during tissue repair. Indeed, cell and extracellular matrix interactions contribute to a number of processes important for tissue healing, including granulation tissue development and tissue remodeling. Cell adhesion plays a role in each of these healing processes and is therefore an important mechanism for the promotion of tissue repair.

Various peptide sequences are important to cell adhesion. For example, the RGD (Arg-Gly-Asp) sequence is an integrin recognition motif found in proteins of the extracellular matrix, such as fibronectin. Integrins are receptors on the cell surface that can cause adhesion between cells and the extracellular matrix by recognizing the RGD motif on the extracellular protein. Indeed, the RGD sequence is able to induce cell adhesion of most cell types with nearly the same affinity as the intact cell adhesion proteins. Thus, when the RGD tripeptide is appropriately coupled to a surface or carrier particle, the tripeptide is capable of reproducing, through integrin binding and clustering, most of the adhesive interactions of RGD sites in the proteins fibronectin, vitronectin, thrombospondin, collagen, laminin, and other extracellular matrix components. Even a very low peptide density, on the order of 10 fmol/cm², is sufficient to support the spreading of cells, clustering of integrin receptors, and organization of actin stress fibers.

### BRIEF SUMMARY

Various embodiments disclosed herein relate to devices, systems, and methods useful for preventing restenosis of blood vessels and repairing blood vessel tissue caused by balloon angioplasty or other injury. For example, disclosed herein are embodiments useful for promoting endothelial regeneration at the site of angioplasty and stent placement. Such endothelialization leads to the repair of the lumen lining and inhibits restenosis by minimizing intimal hyperplasia.

At least some embodiments disclosed herein include a stent for promoting endothelialization, the stent comprising a cylindrical wall and an inner membrane having at least one substantially smooth surface. The cylindrical wall has a first end, a second end, and a lumen extending from the first end to the second end, as well as an external surface and an internal surface. The inner membrane is formed along the internal surface of the cylindrical wall such that the substantially smooth surface of the inner membrane faces the lumen. In addition, the inner membrane may comprise a magnetic particle.

In at least some embodiments, the inner membrane comprises a biological scaffold, an anti-thrombotic agent, and/or small intestinal submucosa.

The cylindrical wall may, in some embodiments, be tapered from the first end of the cylindrical wall to the second end of the cylindrical wall.

In at least certain embodiments, the inner membrane further comprises an endothelialization agent, which may comprise fibrinogen, collagen, and/or a liposome having a magnetic core. In at least some embodiments, the liposome comprises a first layer surrounding the magnetic core, the first layer comprising an amphipathic organic compound, and a second layer surrounding the first layer and the magnetic core, the second layer comprising a tripeptide having an Arg-Gly-Asp sequence. The magnetic core may comprise a magnetic fluid, which may contain Fe₃O₄ (magnetite) or Fe₂O₃. In some embodiments, the amphipathic organic compound of the first layer may comprise one or more of 1,2-Dileoyl-sn-glycero-3-phosphatidylethanolamine, 3beta-N-(N′,N′-dimethylaminoethane)-carbamoyl) cholesterol, egg phosphatidylcholine, N-(a-trimethylammonioacetyl)-didodecyl-D-glutamate chloride, dilauroylphosphatidyl-choline, or dioleoylphosphatidylethanolamine-N-[3-(2-pyridyldithio)-propionate]. The second layer may comprise polyethyleneglycol.

Certain embodiments may have, in addition to an inner membrane, an outer membrane formed along the external surface of the cylindrical wall. The outer member may be attached to the inner membrane at the first and second ends of the cylindrical wall, such that the cylindrical wall is enclosed by the inner and outer membranes.

At least some embodiments disclosed herein include a system for promoting endothelialization in a patient, the system comprising: a stent for implantation into the patient, the stent having (i) a cylindrical wall having a first end, a second end, and a lumen extending from the first end to the second end, the cylindrical wall having an external surface and an internal surface, (ii) an inner membrane having at least one substantially smooth surface, and (iii) a magnetic particle attached to the stent; and an endothelialization agent for administration to the patient; wherein the inner membrane is formed along the internal surface of the cylindrical wall such that the substantially smooth surface of the inner membrane faces the lumen. In certain embodiments, the magnetic particle is impregnated in the inner membrane.

In at least some embodiments, the endothelialization agent comprises a magnetized progenitor cell, which may comprise a magnetic particle and a progenitor cell harvested from the patient.

In at least some embodiments, the cylindrical wall is tapered from the first end of the cylindrical wall to the second end of the cylindrical wall. For the delivery of such embodiments, a system may further include a catheter comprising a balloon, the balloon tapering from a wider end to a narrower end and configured for delivery of the stent.

Various embodiments disclosed herein include a method for promoting endothelialization in a patient, comprising: harvesting a progenitor cell from the patient; combining the progenitor cell and a magnetic particle to form a magnetized progenitor cell; providing a stent comprising (i) a cylindrical wall having a first end, a second end, and a lumen extending from the first end to the second end, the cylindrical wall having an external surface and an internal surface; (ii) an inner membrane having a substantially smooth surface; and (iii) a magnetic particle; wherein the inner membrane is formed along the internal surface of the cylindrical wall such that the substantially smooth surface of the inner membrane faces the lumen; implanting the stent into the patient at a treatment site; and administering the magnetized progenitor cell to the patient. The step of combining the progenitor cell and the magnetic particle may include inducing the progenitor cell to engage in the endocytosis of the magnetic particle.

In at least some embodiments, the magnetic particle is attached to the cylindrical wall or is attached to the inner membrane. It may comprise a liposome having a magnetic core.

The progenitor cell used in certain embodiments may comprise a plurality of progenitor cells, the magnetic particle may comprise a plurality of magnetic particles, and the magnetized progenitor cell may comprise a plurality of magnetized progenitor cells, such that the step of combining the progenitor cell and a magnetic particle to form a magnetized progenitor cell may comprise, in some embodiments, combining the plurality of progenitor cells and the plurality of magnetic particles to form a plurality of magnetized progenitor cells and a plurality of nonmagnetized progenitor cells.

Certain methods may further comprise the step of separating the plurality of magnetized progenitor cells from the plurality of nonmagnetized progenitor cells.

With certain embodiments, such as those having a cylindrical wall that is tapered from the first end of the cylindrical wall to the second end of the cylindrical wall, the step of implanting the stent into the patient at a treatment site may comprise placing the stent using a catheter comprising a balloon, the balloon tapering from a wider end to a narrower end.

At least some embodiments of methods for promoting endothelialization may comprise providing a stent having (i) a cylindrical wall having a first end, a second end, and a lumen extending from the first end to the second end, the cylindrical wall having an external surface and an internal surface; (ii) an inner membrane having a substantially smooth surface; and (iii) a magnetic particle; wherein the inner membrane is formed along the internal surface of the cylindrical wall such that the substantially smooth surface of the inner membrane faces the lumen. The stent is implanted into a patient at a treatment site. In addition, the method may include the steps of preparing an endothelialization agent capable of promoting endothelial cell proliferation and administering the endothelialization agent to the patient.

In at least some embodiments, the endothelialization agent comprises a liposome having a magnetic core. Various embodiments of liposome may further include a first layer surrounding the magnetic core, the first layer comprising an amphipathic organic compound, and a second layer surrounding the first layer and the magnetic core, the second layer comprising a tripeptide having an Arg-Gly-Asp sequence. The magnetic core may comprise a magnetic fluid, and the magnetic fluid may comprise Fe₃O₄ and/or Fe₂O₃.

In certain embodiments, the step of preparing an endothelialization agent may further comprise: (i) dissolving an amount of a phospholipid in an organic solvent; (ii) removing the organic solvent by evaporation, thereby creating a dried film; (iii) combining an amount of iron oxide and an amount of an aqueous fluid; (iv) suspending the dried film in the aqueous fluid by mixing the aqueous fluid and the dried film, thereby forming a suspension of magnetic liposomes and nonmagnetic liposomes; (v) emulsifying the suspension by sonication to form an emulsion; (vi) decanting the emulsion to separate out the magnetic liposomes and the nonmagnetic liposomes; (vii) collecting the magnetic liposomes by magnetic induction; (viii) coupling a tripeptide having an Arg-Gly-Asp sequence with an amount of polyethyleneglycol to form a tripeptide conjugate; and (ix) mixing an amount of the magnetic liposomes with the tripeptide conjugate. The phospholipid may comprise 1,2-Dileoyl-sn-glycero-3-phosphatidylethanolamine, the organic solvent may comprise chloroform, the iron oxide may comprise Fe₃O₄, and the aqueous solution may comprise saline solution. In certain embodiments, the amphipathic organic compound of the first layer may comprise 1,2-Dileoyl-sn-glycero-3-phosphatidylethanolamine, 3beta-N-(N′,N′-dimethylaminoethane)-carbamoyl) cholesterol, and/or egg phosphatidylcholine. The second layer may further comprise polyethyleneglycol. The organic solvent may comprise chloroform, the iron oxide may comprise Fe₃O₄, and the aqueous fluid may consist of water.

In at least some embodiments, the step of dissolving an amount of a phospholipid in an organic solvent comprises dissolving an amount of dioleoylphosphatidylethanolamine-N-[3-(2-pyridyldithio)-propionate] in the organic solvent, dissolving an amount of N-(a-trimethylammonioacetyl)-didodecyl-D-glutamate chloride in the organic solvent, and dissolving an amount of dilauroylphosphatidyl-choline in the organic solvent.

In some embodiments, the step of removing the organic solvent by evaporation may comprise evaporating with dry nitrogen.

Further, in embodiments where the cylindrical wall is tapered from the first end of the cylindrical wall to the second end of the cylindrical wall, the step of implanting the stent into the patient at a treatment site may comprise placing the stent using a catheter comprising a balloon, the balloon tapering from a wider end to a narrower end.

### BRIEF DESCRIPTION OF THE DRAWINGS

FIG. 1 shows an embodiment of a stent for promoting endothelialization, as disclosed herein, within a partially cutaway vessel;

FIG. 2 shows a cross-sectional view of an embodiment of an RGD-magnetic liposome complex as disclosed herein;

FIG. 3 shows an enlarged, partial cross-sectional view of the lipid bilayer of the RGD-magnetic liposome complex embodiment shown in FIG. 2;

FIG. 4 shows a cross-sectional view of another embodiment of stent as disclosed herein;

FIG. 5 shows a side view of another embodiment of a stent for promoting endothelialization, as disclosed herein, within a cutaway vessel; and

FIG. 6 shows an embodiment of a balloon catheter that can be used to deploy a stent such as the stent shown in FIG. 5.

### DETAILED DESCRIPTION

It will be appreciated by those of skill in the art that the following detailed description of the disclosed embodiments is merely exemplary in nature and is not intended to limit the scope of the appended claims.

The disclosed embodiments include devices, systems, and methods useful for preventing restenosis of blood vessels and repairing vessel tissue caused by balloon angioplasty or other injury. For example, disclosed herein are embodiments useful for promoting endothelial regeneration at the site of angioplasty and stent placement. Such endothelialization leads to the repair of the lumen lining of the vessel and inhibits restenosis by minimizing intimal hyperplasia.

When a stent is placed in a vessel, the struts of the stent can cause local blood flow disturbances. Such flow disturbances can inhibit the regeneration of the endothelial lining of the vessel lumen after angioplasty by affecting endothelial biology and function. Consequently, blood flow disturbances have been implicated in restenosis. For example, flow disturbances have been linked to the development of intimal hyperplasia, which can cause restenosis, especially in smaller diameter vessels.

Covering the stent with an inner membrane can reduce the local flow disturbances by shielding the flowing blood from the stent struts. Endothelialization is further enhanced, however, by promoting the growth of endothelial cells on the membrane so that the stent is effectively hidden from the flowing blood within the wall of the vessel. This can be accomplished in several ways, including by directing endothelial progenitor cells to the targeted site using magnetic or other means.

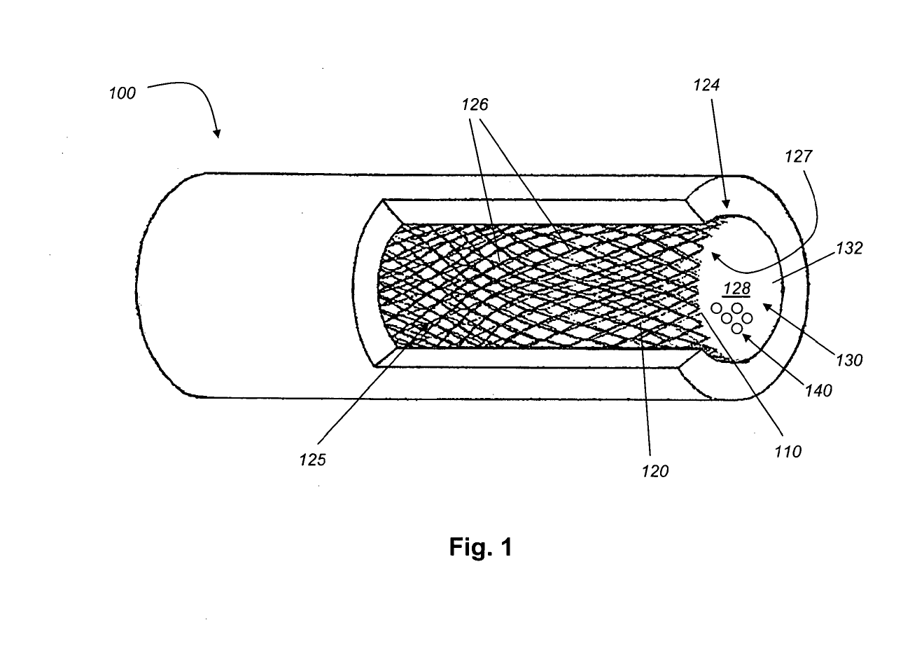

Referring now to FIG. 1, there is shown blood vessel 100, which is partially cutaway to reveal a stent 110 for promoting endothelialization inside vessel 100. Stent 110 comprises a curtain-like cylindrical wall 120 having a first end 124, a second end (not shown), and a lumen 128 extending from first end 124 to the second end (not shown). Cylindrical wall 120 further comprises an external surface 125 and an internal surface 127, which faces lumen 128. Cylindrical wall 120 has a generally circular cross-sectional shape, but other embodiments of cylindrical walls may have cross-sections of any suitable shape, such as an oval or octagon shape. Indeed, at least some embodiments have a cylindrical wall that tapers from a wider end to a narrower end, as is disclosed herein (see FIG. 5).

Cylindrical wall 120 is shown having an expandable wire form, which comprises struts 126. However, other embodiments of cylindrical walls may comprise a perforated tube; such tubes are conventionally made by laser cutting. Almost any material is suitable for making the cylindrical wall, including stainless steel, titanium, or nitinol, so long as the material is biocompatible and the stent can be deployed in a contracted position and expanded in the lumen. In at least some embodiments, the cylindrical wall is flexible. Indeed, in at least certain embodiments, the cylindrical wall, and therefore the stent, need not provide substantial support to the vessel. Rather, such stents prevent restenosis by promoting endothelialization and by preventing turbulent blood flow. Therefore, such stents avoid the problems caused by excessive rigidity of the stent struts (e.g., compliance mismatch). Stents made from memory-forming materials may be used.

Moreover, proper stent sizing may be accomplished using an impedance catheter, as disclosed in International Patent Application No. PCT/US2004/004828, filed Feb. 18, 2004 (Publ. No. WO 2004/75928).

Referring again to FIG. 1, stent 110 further comprises an inner membrane 130 having at least one substantially smooth surface 132. Inner membrane 130 is a biocompatible membrane that is formed along internal surface 127 of cylindrical wall 120 such that substantially smooth surface 132 faces lumen 128. An inner membrane, such as inner membrane 130, may be comprised of silicon or any other suitable material that is stretchable, permeable, and allows for endothelialization. In addition, the inner membrane should have a substantially smooth surface, such as substantially smooth surface 132. Substantially smooth surface 132 need only be smooth enough to prevent blood flow disturbances caused by the presence of the cylindrical wall and to be conducive to endothelialization. In other words, the substantially smooth surface directs blood flow around the struts of a stent without creating significant turbulence.

On the opposing surface of the inner membrane, a drug or other chemical may be attached for delivery to the vessel wall. Indeed, the inner membrane may shield the blood stream from the release of drugs (or other chemical agents) from the opposing surface of the inner membrane or from the cylindrical wall. For example, the opposing surface of the inner membrane may comprise an anti-proliferative. Moreover, a drug or other chemical agent may be embedded within the inner membrane, which provides a substantial volume and surface area available for delivery of the drug. Multiple drugs having different release rates may also be released from the inner membrane.

At least some embodiments of inner membrane may comprise a biological scaffold (which may be seeded with collagen or fibrinogen), or may comprise small intestinal submucosa (“SIS”). The scaffold or SIS may be coated with an anti-thrombotic agent (e.g., heparin or anti-platelets) to prevent thrombosis or other problems caused by coagulation of the blood. Alternatively, the patient may be treated with an anti-thrombotic regimen to help prevent such problems.

Before insertion into a patient, the stent is collapsed and the inner membrane or scaffold is compressed or wrinkled. Upon deployment or expansion of the stent, the inner membrane or scaffold stretches to its original size.

Referring again to FIG. 1, inner membrane 130 comprises magnetic particles 140. Magnetic particles 140 may be impregnated in the inner membrane or otherwise attached to the surface of the inner membrane (such as substantially smooth surface 132 of inner membrane 130). However, in at least some other embodiments, the magnetic particles may be attached to the cylindrical wall. Magnetic particles 140 may comprise particles of any suitable magnetic material, including permanent magnets and paramagnets, such as iron oxide. In addition, in at least some embodiments, the magnetic particles may comprise a liposome that has a magnetic core.

Referring again to FIG. 1, inner membrane 130 may further comprise an endothelialization agent. Endothelial agents have some capability of promoting endothelial growth and are used to enhance endothelialization at the treatment site. Suitable embodiments of endothelialization agents may comprise, for example, progenitor endothelial cells, liposomes comprising an endothelialization promoter (such as the RGD (Arg-Gly-Asp) tripeptide sequence), or collagen, fibrinogen, or another suitable biological proteins.

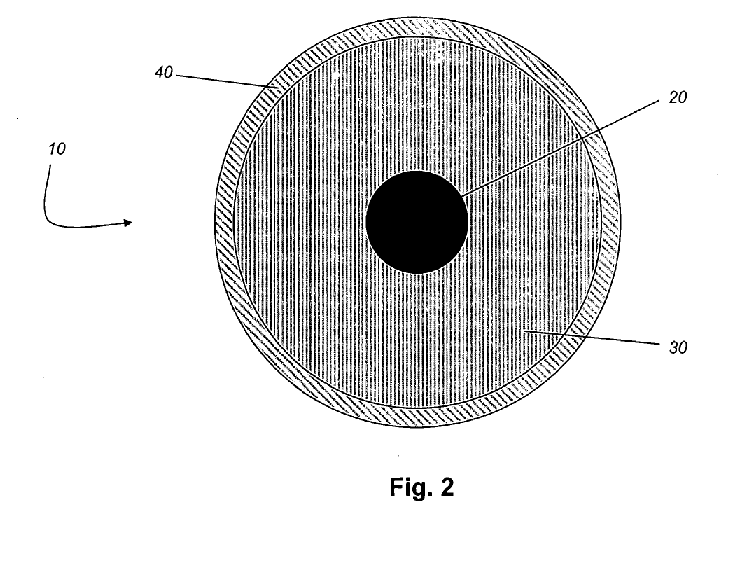

For example, referring now to FIG. 2, there is shown liposome 10, which includes a magnetic core 20, a first layer 30, and a second layer 40. Magnetic core 20 comprises a magnetic fluid having a number of nanoparticles of Fe₃O₄, which is a form of iron oxide known as magnetite. Although magnetic core 20 comprises magnetite, at least some other embodiments of magnetic cores comprise other types of iron oxide, such as Fe₂O₃, or rare earth metals. However, the particles should be easily magnetized with an external magnetic field and should be easily dispersed once the magnet is removed. In this way, although the core is typically referred to as a magnetic core, the core may be understood to be a paramagnetic or super-paramagnetic core. Further, while magnetic core 20 comprises a magnetic fluid, which is a suspension of magnetic or paramagnetic nanoparticles in a carrier liquid, at least some other types of magnetic cores do not comprise a carrier liquid (and therefore do not comprise a magnetic fluid). Such magnetic liposomes may be prepared by entrapment of ferrofluids within the core of the liposome, as is discussed herein.

The nanoparticles of iron oxide may be of any suitable size, but generally have a mean particle size of 10 microns. As explained below, the particles will be surrounded by first layer 30, which will form a liposome around magnetic core 20. Therefore, the particles should be small enough to fit within the magnetic core inside the liposome.

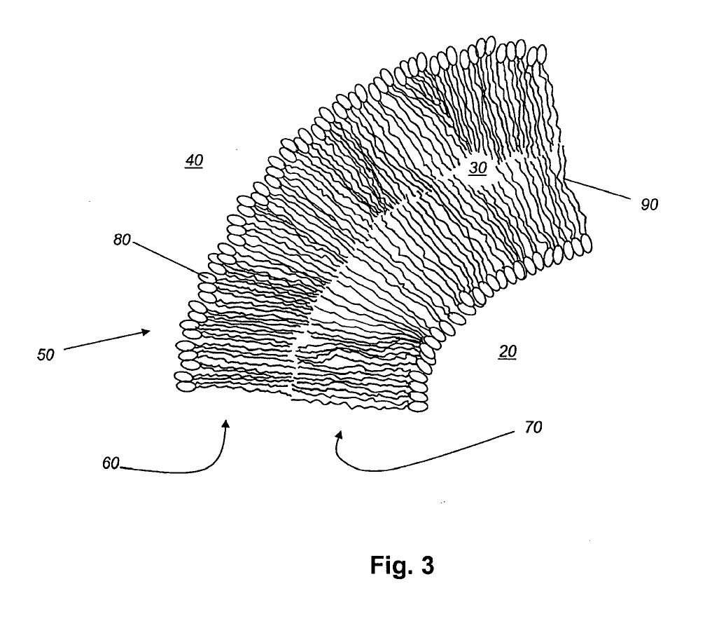

Referring again to FIG. 2, first layer 30 surrounds magnetic core 20 and comprises an amphipathic organic compound comprising 1,2-Dileoyl-sn-glycero-3-phosphatidylethanolamine and 3beta-N-(N′,N′-dimethylaminoethane)-carbamoyl) cholesterol. Other suitable amphipathic organic compounds may comprise egg phosphatidylcholine, N-(a-trimethylammonioacetyl)-didodecyl-D-glutamate chloride, dilauroylphosphatidyl-choline, dioleoylphosphatidylethanolamine-N-[3-(2-pyridyldithio)-propionate], or any combination thereof. Indeed, the first layer surrounding the magnetic core may comprise any suitable amphipathic organic compound that is biocompatible, that is capable of forming a liposome around the magnetic core, and that is capable of binding to the RGD peptide (as discussed below). A liposome is a hollow nanoparticle comprising an external lipid bilayer. Liposomes are generally biocompatible, non-immunogenic, and non-inflammatory. Referring now to FIG. 3, there is shown a partial cross-sectional view of first layer 30, showing lipid bilayer 50. Lipid bilayer 50 comprises an external layer 60 of amphipathic lipid molecules and an internal layer 70 of amphipathic lipid molecules. Each of the amphipathic molecules has a polar head 80 and one or more nonpolar tails 90. Magnetic core 20 is therefore surrounded by lipid bilayer 50 of first layer 30. The liposomes generally have a positive charge on their surfaces.

Referring again to FIG. 2, second layer 40 surrounds first layer 30 and magnetic core 20. Second layer 40 comprises a tripeptide having an Arg-Gly-Asp sequence (“RGD peptide”). RGD-containing peptides may be grafted to any of a number of polymers, including polymer-modified glass substrate, biodegradable copolymer of polylysine and polyactic acid, hydrogels, polyacrylamide, polyurethane, polyvinyl alcohol, and polyethylene glycol. In the embodiment shown in FIG. 2, RGD peptides are coupled to polyethylene glycol, therefore second layer 40 comprises polyethylene glycol.

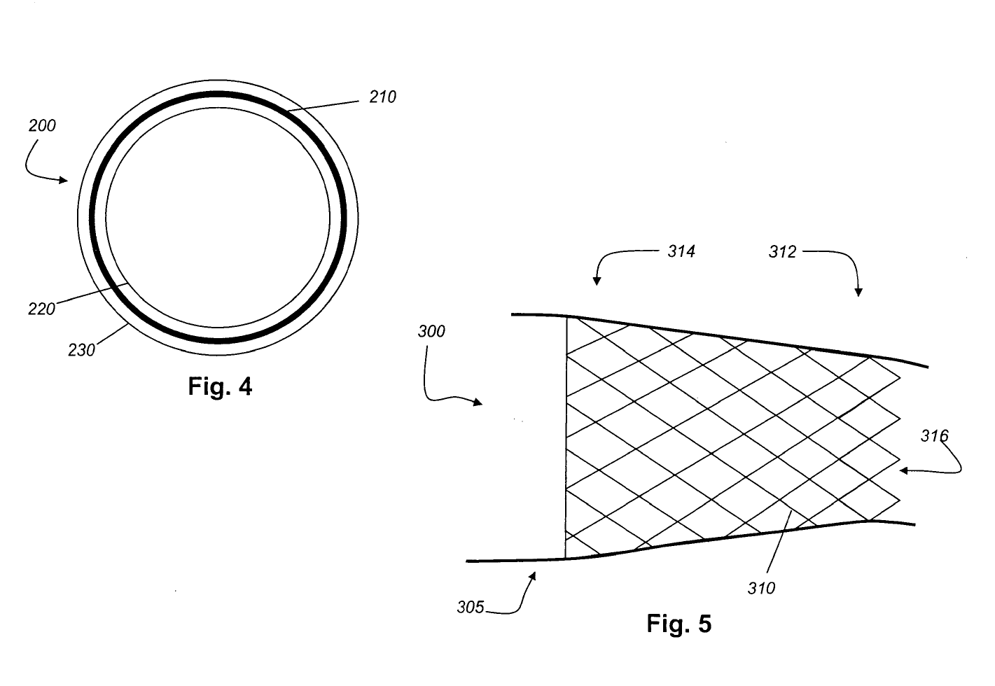

Stent 110 (shown in FIG. 1) comprises cylindrical wall 120 and inner membrane 130. However, other embodiments of stents may further comprise additional layers. For example, referring to FIG. 4, there is shown a cross-sectional view of stent 200. Stent 200 comprises cylindrical wall 210, inner membrane 220, and outer membrane 230. Cylindrical wall 210 is similar to cylindrical wall 120 of stent 110 (shown in FIG. 1), and inner membrane 220 is similar to inner membrane 130 of stent 110 (shown in FIG. 1). Outer membrane 230 is formed along the external surface of cylindrical wall 210. Outer membrane 230 is attached to inner membrane 220 at the first and second ends (not shown) of stent 200, such that cylindrical wall 210 is enclosed by inner membrane 220 and outer membrane 230. Cylindrical wall 210 is attached to inner membrane 220 and outer membrane 230, but at least certain other embodiments have a cylindrical wall that is not attached to either the inner membrane or the outer membrane.

Outer membrane 230 is made of the same material as inner membrane 220. However, at least some other embodiments have an outer membrane that is not made of the same material as the inner membrane, so long as the outer membrane is made of a suitable material, such as silicon or any other suitable material that is stretchable and permeable. Outer membrane 230 may comprise an anti-thrombotic agent on its abluminal surface.

Referring now to FIG. 5, there is shown a stent 300 placed within a vessel wall 305. Stent 300 comprises a cylindrical wall 310 having a first end 312, a second end 314, and a lumen 316 extending from first end 312 to second end 314. As shown, cylindrical wall 310 is tapered from wider second end 314 to narrower first end 312. This taper helps reduce local blood flow turbulence by making the stent more hemodynamic, as compared to non-tapered stents.

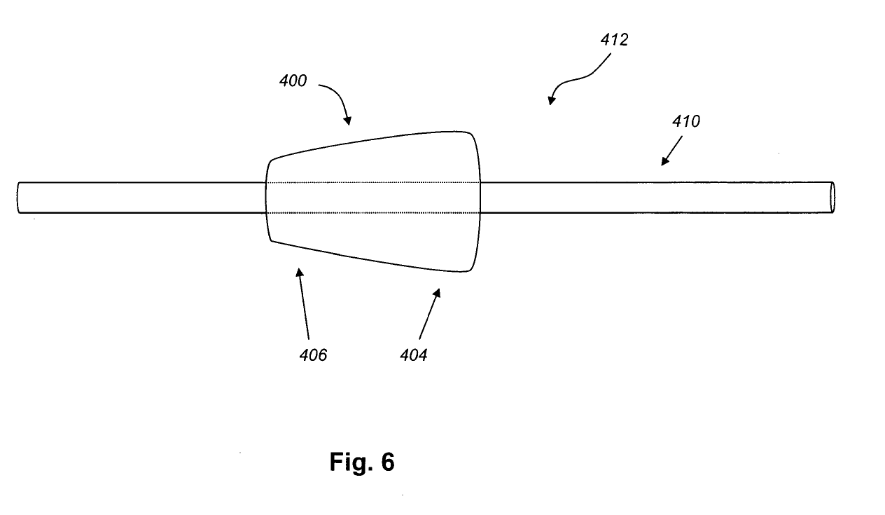

Stent 300 may be placed using an angioplasty balloon that expands to a greater degree at one end than the other end. For example, referring to FIG. 6, there is shown a balloon 400 attached to a balloon catheter 410 at a distal end 412 of catheter 410. When expanded, as shown, balloon 400 tapers from a wide end 404 to a narrow end 406. This tapered balloon is effective for placing tapered stents, such as stent 300, into a vessel and expanding the stent to the proper dimensions.

At least some embodiments disclosed herein are administered to patients in a number of ways. For example, a stent (such as stent 110 of FIG. 1) may be implanted into a patient at a treatment site, such as a blood vessel (see, e.g., FIG. 1), which may be a central vessel or a peripheral vessel, or any other suitable body lumen. The clinician may also administer an endothelialization agent, such as, for example, magnetized progenitor cells. In such embodiments, a clinician may harvest a number of progenitor endothelial cells from the patient by separating the cells from other blood cells after withdrawing the patient's blood. Progenitor cells harvested from the patient are preferred because they generally have maximum compatibility with the patient and therefore reduce the risk of donor-host rejection.

After harvest, the progenitor cells are combined with magnetic particles, such as ferrous particles, to form magnetized progenitor cells. The cells can be combined with the magnetic particles by inducing the progenitor cells to engage in the endocytosis of the particles. Endothelial cells are incubated with the magnetic particles, which become internalized by the cells. However, in most cases, at least some progenitor cells do not combine with magnetic particles, so both magnetized progenitor cells and nonmagnetized progenitor cells will remain after the combination process. In at least some cases, the clinician may separate the magnetized progenitor cells from the nonmagnetized progenitor cells by applying a magnetic field to the cells and physically separating the cells that respond to the magnetic field (e.g., induction). However, separation of the magnetized progenitor cells from the nonmagnetized progenitor cells is not necessary in all cases.

The clinician then administers the magnetized progenitor cells (and potentially some nonmagnetized progenitor cells) to the patient, for example, by intravenous injection. In embodiments of stents comprising a magnetic particle, the magnetized progenitor cells are drawn to and collect near the stent (and specifically the magnetic particle). Thus, for example, a stent (such as stent 110 of FIG. 1) may comprise an inner membrane (such as inner membrane 130 of FIG. 1) having a plurality of magnetic particles attached—either impregnated in the membrane or attached to the surface of the membrane. Because of their magnetic interaction with the magnetic particles, the magnetized progenitor cells will collect along the inner membrane and will then begin to lay down an epithelial layer on the substantially smooth surface of the inner membrane. This new epithelial layer will tend to inhibit intimal hyperplasia and decrease further the blood flow disturbances at the site of the stent placement.

In at least some embodiments, the clinician may administer a different endothelialization agent. For example, the clinician may administer an amount of an RGD-liposome complex (see, e.g., FIG. 2) in a biocompatible carrier fluid. In such a case, multiple RGD-liposome complexes are suspended in the carrier fluid and may be administered to a human patient by intravenous injection, or by any other suitable method, including local or systemic administration.

For example, a clinician may implant a stent (such as stent 110 of FIG. 1) into a patient at a treatment site. An endothelialization agent capable of promoting endothelial cell proliferation is prepared and administered to the patient. The endothelialization agent comprises a liposome such as liposome 10 of FIG. 2, which has a magnetic core 20, a first layer 30 surrounding the magnetic core, the first layer comprising an amphipathic organic compound, and a second layer 40 surrounding the first layer and the magnetic core, the second layer comprising a tripeptide having an Arg-Gly-Asp (RGD) sequence. In at least some embodiments, the magnetic core comprises a magnetic fluid, which may comprise iron oxide, such as Fe₃O₄ or Fe₂O₃, or a rare earth metal. The RGD-magnetic liposome complexes are attracted to and collect at the site of the stent placement, due to the magnetic interaction between the magnetic core of the liposome complex and the magnetic particles on the stent. The RGD tripeptides then promote cell adhesion and endothelialization in and around the stent.

The endothelialization agent may be prepared in a number of ways. For instance, an amount of phospholipid may be dissolved in an organic solvent. The phospholipid may comprise (i) 1,2-Dileoyl-sn-glycero-3-phosphatidylethanolamine; (ii) 3beta-N-(N′,N′-dimethylaminoethane)-carbamoyl) cholesterol; (iii) egg phosphatidylcholine; (iv) dioleoylphosphatidylethanolamine-N-[3-(2-pyridyldithio)-propionate]; (v) N-(a-trimethylammonioacetyl)-didodecyl-D-glutamate chloride; (vi) dilauroylphosphatidyl-choline; or (vii) any combination thereof. The chosen phospholipid(s) form the amphipathic organic compount of the first layer of the resulting liposome (see, e.g., first layer 30 of liposome 10 shown in FIG. 1).

For example, a 1:2:2 molar ratio of N-(a-trimethylammonioacetyl)-didodecyl-D-glutamate chloride, dilauroylphosphatidyl-choline, and dioleoylphosphatidylethanolamine-N-[3-(2-pyridyldithio)-propionate] may be dissolved in chloroform (at a 10-20 mg of lipid for each ml of solvent ratio) in a round bottom flask.

The organic solvent may comprise chloroform, diethyl ether, or polyethylene glycol. Other suitable phospholipids and organic solvents may be used.

The organic solvent is removed, usually by evaporation, creating a dried film. Evaporation may occur using dry nitrogen, a rotary evaporator, or any other suitable method. To ensure all organic solvent is evaporated from the lipid layer, the flask may be placed on a vacuum pump overnight.

Iron oxide is combined in an amount of aqueous fluid, such as saline solution or water. The iron oxide nanoparticles may comprise any type of iron oxide, such as Fe₂O₃ or Fe₃O₄. The dried film is suspended in the aqueous fluid by mixing the dried film and the aqueous fluid, forming a suspension of magnetic liposomes and nonmagnetic liposomes. Vortex mixing may be used.

The suspension may be emulsified by sonication to form an emulsion. For example, the suspension can be sonicated for about 30 minutes at 28W. The emulsion may then be decanted to separate out the magnetic liposomes and the nonmagnetic liposomes. The magnetic liposomes can be collected by magnetic induction. Indeed, repeated magnetic induction may be beneficial. Induction at 0.23-0.4 T may be useful.

A tripeptide conjugate is formed by coupling a tripeptide having an Arg-Gly-Asp (RGD) sequence with an amount of polyethyleneglycol. The tripeptide conjugate is then mixed with an amount of the magnetic liposomes to form the RGD-liposome complex. Specifically, the tripeptide and polyethyleneglycol form the second layer of the liposome (such as second layer 40 of liposome 10 shown in FIG. 2).

As another example, 13.4 μmol of 1,2-Dileoyl-sn-glycero-3-phosphatidylethanolamine and 18.6 μmol of 3beta-N-(N′,N′-dimethylaminoethane)-carbamoyl) cholesterol may be dissolved in 3 ml of chloroform. Alternatively, a solution of egg phosphatidylcholine (26 μmol) and cholesterol (26 μmol) may be dissolved in 3 ml of diethyl ether. The organic solvent is then removed using a rotary evaporator, leaving a dried film. By shaking vigorously with a vortex mixer, the dried film is suspended in 1 ml of 0.9% saline solution containing 10 mg of magnetite particles. After 24 hours of hydration at 4° C., the suspension is emulsified by sonication for 60 minutes in a bath sonicator (ultrasonic cleaner). The magnetic liposomes are then collected. As a further alternative, magnetite and a solution of 1,2-Dilinoleoyl-3-palmitoyl-rac-glycerol may be added to a saline buffer solution (e.g., Hank's Balanced Salt Solution (HBSS), Invitrogen Corp.) and then incubated at room temperature for 60 minutes.

To form the RGD-magnetic liposomes, cyclic RGD peptides (100 μmol) may be coupled to the succinimidyl carboxyester terminus of a polyethyleneglycol (3400) by reacting the RGD peptides with 10 times the molar excess (1 mmol) of polyethyleneglycol in 8 ml of N,N-dimethylformamide at room temperature for 2 hours. Two milliliters of magnetic liposomes may then be mixed with RGD-polyethyleneglycol conjugates to give an approximate molar ratio of 0.55 of peptides to 1 of liposomes. The coupling is facilitated by using gentle agitation at room temperature for about 3 to 3.5 hours in morpholinoethanesulfonic acid (a buffer).

The resulting mixture may be further separated by placing the mixture in a magnetic stand and vacuuming out the free particles (i.e., those that do not adhere to the sides). The remaining nanoparticles in suspension should thereafter precipitate out.

While various embodiments of devices, systems, and methods for promoting endothelialization have been described in considerable detail herein, the embodiments are merely offered by way of non-limiting examples of the invention described herein. Many variations and modifications of the embodiments described herein will be apparent to one of ordinary skill in the art in light of the this disclosure. It will therefore be understood by those skilled in the art that various changes and modifications may be made, and equivalents may be substituted for elements thereof, without departing from the scope of the invention. Indeed, this disclosure is not intended to be exhaustive or to limit the scope of the invention. The scope of the invention is to be defined by the appended claims, and by their equivalents.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present invention.

It is therefore intended that the invention will include, and this description and the appended claims will encompass, all modifications and changes apparent to those of ordinary skill in the art based on this disclosure.

1. A stent for promoting endothelialization, comprising:
a cylindrical wall having a first end a second end, and a lumen extending from the first end to the second end, the cylindrical wall having an external surface and an internal surface; and
an inner membrane having at least one substantially smooth surface, the inner membrane comprising a magnetic particle.
wherein the inner membrane is formed along the internal surface of the cylindrical wall such that the substantially smooth surface of the inner membrane faces the lumen.

2. The stent of claim 1, wherein:
the inner membrane further comprises an endothelialization agent.

3. The stent of claim 23 wherein:
the endothelialization agent is at least one agent chosen from fibrinogen or collagen.

4. (canceled)

5. The stent of claim 2, wherein:
the endothelialization agent comprises a liposome having a magnetic core.

6. The stent of claim 5, wherein:
the liposome further comprises a first layer surrounding the magnetic core. the first layer comprising an amphipathic organic compound, and a second layer surrounding the first layer and the magnetic core, the second layer comprising a tripeptide having an Arg-Gly-Asp sequence.

7. The stent of claim 6, further comprising:
an outer membrane formed along the external surface of the cylindrical wall, the outer member attached to the inner membrane at the first and second ends of the cylindrical wall, such that the cylindrical wall is enclosed by the inner and outer membranes.

8. The stent of claim 6, wherein:
the magnetic core comprises a magnetic fluid.

9. The stent of claim 8, wherein:
the magnetic fluid is at least one fluid chosen from Fe₃O₄ or Fe₂O₃.

10. (canceled)

11. The stent of claim 6, wherein:
the amphipathic organic compound of the first layer is at least one compound chosen from 1,2-Dileoyl-sn-glycero-3-phosphatidylethanolamine, 3beta-N-(N′N′-dimethylaminoethane)carbamoyl) cholesterol egg phosphatidylcholine, N-(a-trimethylammonioacetyl)-didodecyl-D-glutamate chloride, dilauroylphosphatidyl-choline, or diolcoylphosphatidylethanolamine-N-[3-(2-pyridyldithio)-propionate].

12-16. (canceled)

17. The stent of claim 6, wherein:
the amphipathic organic compound of the first layer comprises N-(a-trimethylammonioacetyl)-didodecyl-D-glutamate chloride, dilauroylphosphatidyl-choline and dioleoylphosphatidylethanolamine-N-[3-(2-pyridyldithio)-propionate].

18. The stent of claim 6, wherein:
the second layer further comprises polyethyleneglycol.

19. The stent of claim 65 further comprising:
the magnetic core comprises a magnetic fluid;
the amphipathic organic compound of the first layer comprises 1,2-Dileoyl-sn-glycero-3-phosphatidylethanolamine and 3beta-N-(N′,N′-dimethylaminoethane)-carbamoyl) cholesterol; and
the second layer further comprises polyethyleneglycol.

20. The stent of claim 6, wherein:
the magnetic core comprises Fe₃O₄; and
the amphipathic organic compound of the first layer comprises N-(a-trimethylammonioacetyl)-didodecyl-D-glutamate chloride, dilauroylphosphatidyl-choline, and dioleoylphosphatidylethanolamine-N-[3-(2-pyridyldithio)-propionate].

21. The stent of claim 6, wherein:
the inner membrane further comprises a biological scaffold.

22. The stent of claim 6, wherein,
the inner membrane further comprises an anti-thrombotic agent.

23. The stent of claim 6, wherein:
the inner membrane further comprises small intestinal submucosa.

24. The stent of claim 1, wherein:
the cylindrical wall is tapered from the first end of the cylindrical wall to the second end of the cylindrical wall.

25. A system for promoting endothelialization in a patient, comprising;
a stent for implantation into the patient, the stent comprising (i) a cylindrical wall having a first end, a second end, and a lumen extending from the first end to the second end, the cylindrical wall having an external surface and an internal surface; (ii) an inner membrane having at least one substantially smooth surface; and (iii) a magnetic particle attached to the stent; and
an endothelialization agent for administration to the patient;
wherein the inner membrane is formed along the internal surface of the cylindrical wall such that the substantially smooth surface of the inner membrane faces the twice.

26. The system of claim 25, wherein:
the magnetic particle is impregnated in the inner membrane.

27. The system of claim 26, wherein:
the endothelialization agent comprises a magnetized progenitor cell,

28. The system of claim 27, wherein:
the magnetized progenitor cell comprises a magnetic particle and a progenitor cell harvested from the patient.

29. The system of claim 25, wherein:
the stent further comprises an outer membrane formed along the external surface of the cylindrical wall, the outer member attached to the inner membrane at the first and second ends of the cylindrical wall, such that the cylindrical wall is enclosed by the inner and outer membranes.

30. The system of claim 25, wherein:
the cylindrical wall is tapered from the first end of the cylindrical wall to the second end of the cylindrical wall.

31. The system of claim 30, further comprising:
a catheter comprising a balloon, the balloon tapering from a wider end to a narrower end and configured for delivery of the stent.

32. The system of claim 25, wherein:
the endothelialization agent comprises a liposome having a magnetic core, a first layer surrounding the magnetic core, the first layer comprising an amphipathic organic compound, and a second layer surrounding the first layer and the magnetic core, the second layer comprising a tripeptide having an Arg-Gly-Asp sequence.

33. The system of claim 32, wherein:
the magnetic core comprises a magnetic fluid;
the amphipathic organic compound of the first layer comprises 1,2-Dileoyl-sn-glycero-3-phosphatidylethanolamine and 3beta-N-(N′,N′-dimethylaminoethane)-carbamoyl) cholesterol; and
the second layer further comprises polyethyleneglycol.

34. The system of claim 33, wherein:
the magnetic core further comprises magnetite.

35. A method for promoting endothelialization in a patient, comprising:
harvesting a progenitor cell from the patient;
combining the progenitor cell and a magnetic particle to form a magnetized progenitor cell;
providing a stent comprising (i) a cylindrical wall having a first end, a second end, and a lumen extending from the first end to the second end, the cylindrical wall having an external surface and an internal surface; (ii) an inner membrane having a substantially smooth surface; and (iii) a magnetic particle; wherein the inner membrane is formed along the internal surface of the cylindrical wall such that the substantially smooth surface of the inner membrane faces the lumen;
implanting the stent into the patient at a treatment site; and
administering the magnetized progenitor cell to the patient.

36. The method of claim 35, wherein:
the magnetic particle is attached to the cylindrical wall.

37. The method of claim 35, wherein:
the magnetic particle is attached to the inner membrane.

38. The method of claim 35, wherein:
the step of combining the progenitor cell and the magnetic particle comprises inducing the progenitor cell to engage in the endocytosis of the magnetic particle.

39. The method of claim 38, wherein:
the progenitor cell comprises a plurality of progenitor cells;
the magnetic particle comprises a plurality of magnetic particles; and
the magnetized progenitor cell comprises a plurality of magnetized progenitor cells;
such that the step of combining the progenitor cell and a magnetic particle to form a magnetized progenitor cell comprises combining the plurality of progenitor cells and the plurality of magnetic particles to form a plurality of magnetized progenitor cells and a plurality of nonmagnetized progenitor cells.

40. The method of claim 39, further comprising:
separating the plurality of magnetized progenitor cells from the plurality of non-magnetized progenitor cells,

41. The method of claim 35, wherein:
the magnetic particle comprises a liposome having a magnetic core.

42. The method of claim 41, wherein:
the liposome further comprises a first layer surrounding the magnetic core, the first layer comprising an amphipathic organic compound, and a second layer surrounding the first layer and the magnetic core, the second layer comprising a tripeptide having an Arg-Gly-Asp sequence.

43. The method of claim 42, wherein:
the magnetic core comprises magnetite.

44. The method of claim 43, wherein:
the amphipathic organic compound of the first layer comprises 1,2-Dileoyl-sn-glycero-3-phosphatidylethanolamine.

45. The method of claim 35, wherein:
the cylindrical wall is tapered from the first end of the cylindrical wall to the second end of the cylindrical wall.

46. The method of claim 45, wherein:
the step of implanting the stent into the patient at a treatment site comprises placing the stent using a catheter comprising a balloon, the balloon tapering from a wider end to a narrower end.

47. A method for promoting endothelialization, comprising:
providing a stent comprising (i) a cylindrical wall having a first end, a second end, and a lumen extending from the first end to the second end, the cylindrical wall having an external surface and an internal surface; (ii) an inner membrane having a substantially smooth surface; and (iii) a magnetic particle; wherein the inner membrane is formed along the internal surface of the cylindrical wall such that the substantially smooth surface of the inner membrane faces the lumen;
implanting the stent into a patient at a treatment site;
preparing an endothlelialization agent capable of promoting endothelial cell proliferation; and
administering the endothelialization agent to the patient.

48. The method of claim 47, wherein:
the endothelialization agent comprises a liposome having a magnetic core.

49. The method of claim 48, wherein:
the liposome further comprises a first layer surrounding the magnetic core, the first layer comprising an amphipathic organic compound, and a second layer surrounding the first layer and the magnetic core, the second layer comprising a tripeptide having an Arg-Gly-Asp sequence.

50. The method of claim 49, wherein:
the magnetic core comprises a magnetic fluid.

51. The method of claim 50, wherein:
the magnetic fluid is at least one fluid chosen from Fe₃O₄ or Fe₂O₃.

52. (canceled)

53. The method of claim 50, wherein:
the step of preparing an endothelialization agent further comprises: (i) dissolving an amount of a phospholipid in an organic solvent; (ii) removing the organic solvent by evaporation, thereby creating a dried film; (iii) combining an amount of iron oxide and an amount of an aqueous fluid; (iv) suspending the dried film in the aqueous fluid by mixing the aqueous fluid and the dried film, thereby forming a suspension of magnetic liposomes and nonmagnetic liposomes; (v) emulsifying the suspension by sonication to form an emulsion; (vi) decanting the emulsion to separate out the magnetic liposomes and the nonmagnetic liposomes; (vii) collecting the magnetic liposomes by magnetic induction; (viii) coupling a tripeptide having an Arg-Gly-Asp sequence with an amount of polyethyleneglycol to form a tripeptide conjugate; and (ix) mixing an amount of the magnetic liposomes with the tripeptide conjugate.

54. The method of claim 53, wherein:
the phospholipid comprises 1,2-Dileoyl-sn-glycero-3-phosphatidylethanolamine;
the organic solvent comprises chloroform;
the iron oxide comprises Fe₃O₄; and
the aqueous solution comprises saline solution.

55. The method of claim 49, wherein:
the amphipathic organic compound of the first layer comprises 1,2-Dileoyl-sn-glycero-3-phosphatidylethanolamine.

56. The method of claim 49, wherein:
the amphipathic organic compound of the first layer comprises 3beta-N-(N′,N′-dimethylaminoethane)-carbamoyl) cholesterol.

57. The method of claim 49, wherein:
the amphipathic organic compound of the first layer comprises egg phosphatidylcholine.

58. The method of claim 49, wherein:
the second layer further comprises polyethyleneglycol.

59. The method of claim 53, wherein:
the step of dissolving an amount of a phospholipid in an organic solvent comprises dissolving an amount of dioleoylphosphatidylethanolamine-N-[3-(2-pyridyldithio)-propionate] in the organic solvent, dissolving an amount of a N-(a-trimethylammonioacetyl)-didodecyl-D-glutamate chloride in the organic solvent; and dissolving an amount of dilauroylphosphatidyl-choline in the organic solvent.

60. The method of claim 59, wherein:
the organic solvent comprises chloroform;
the iron oxide comprises Fe₃O₄; and
the aqueous fluid consists of water.

61. The method of claim 60, wherein:
the step of removing the organic solvent by evaporation comprises evaporating with dry nitrogen.

62. The method of claim 47, wherein:
the cylindrical wall is tapered from the first end of the cylindrical wall to the second end of the cylindrical wall.

63. The method of claim 62, wherein:
the step of implanting the stent into the patient at a treatment site comprises placing the stent using a catheter comprising a balloon, the balloon tapering from a wider end to a narrower end.
